(19) United States
(12) Patent Application Publication
(10) Pub. No.: US 2025/0295847 A1
(43) Pub. Date: Sep. 25, 2025

(54) METHOD AND MACHINE FOR TRANSPLANTING FAT

(71) Applicant: NEOSYAD, AIX-EN-PROVENCE (FR)

(72) Inventors: Régis ROCHE, LORGUES (FR); François CABAUD, EGUILLES (FR); Xavier NELISSEN, VILLERS SAINT SIMEON (BE)

(21) Appl. No.: 18/860,760

(22) PCT Filed: Apr. 27, 2023
(86) PCT No.: PCT/FR2023/000060

§ 371 Date: Oct. 28, 2024

(30) Foreign Application Priority Data
Apr. 29, 2022 (FR) FR2204070

(51) Int. Cl. A61M 1/00; B01D 21/26; B04B 3/00; B04B 9/02; B04B 9/10

(52) U.S. Cl. CPC A61M 1/893; A61M 1/895; B01D 21/262; B04B 3/00; B04B 9/02; B04B 9/10; A61M 2205/3393; A61M 2210/1007

(57) ABSTRACT

The present invention relates to a fat transplantation machine (1) comprising:
a fat suction circuit,
a filtration device (100) comprising a filter delimiting a centrifugation chamber (160) for an adipose tissue, the filter having a pore size configured to allow a liquid medium to pass and retain an adipose tissue,
a rotational drive device (30) connected to the filtration device,
a mass measurement device (5) configured to measure the mass of adipose tissue present in the filtration device,
a processing unit (10) configured to control the rotational drive device (300) so as to initiate at least one centrifugation filtration sequence as a function of a predetermined mass of suctioned adipose tissue, the processing unit being further configured to calculate an injectable amount of adipose tissue (Q_{injectable}) after each purification sequence.

### TECHNICAL FIELD

The present invention relates to a method and a machine for fat transplantation operations (referred to as lipofilling) for aesthetic and reconstructive purposes. The invention may particularly find a use for the transplantation of fat useful for breast surgery operations, the implementation of the device which is the object of the invention however not being limited to this application.

### PRIOR ART

Fat transplantation or autologous adipose tissue grafting operations are used in surgery, in particular in buttock surgery for volume augmentation or in breast surgery in order to shape the breast to give it a more natural appearance after a DIEP (Deep Inferior Epigastric Perforator), latissimus dorsi flap reconstruction, or after breast implant placement. These operations consist in removing fat from a patient in a donor area and then reintroducing it into the area of interest of the patient's body, the removed fat being treated before its reintroduction.

Regardless of the planned transplantation operation, the control of the amount of fat throughout the transplantation process is an important parameter. Indeed, during the liposuction operation, it is important for the practitioner to be able to determine how much fat should be removed to obtain the amount of fat needed for the injection. However, the adipose tissue removed is treated to eliminate in particular the liquid medium and polluting species (physiological fluid, blood, oil, etc.) present in the tissue. It is therefore understood that the removed amount of adipose tissue does not correspond to the potentially injectable amount of tissue.

In the case of breast augmentation or reconstruction, for example, the practitioner determines the volume to be injected during a prior examination in order to obtain an optimal result. Thus, if the practitioner removes by liposuction a smaller amount of adipose tissue than what is necessary, the final result will not be satisfactory.

Furthermore, during the operation of injecting the treated adipose tissue, it is also important for the practitioner to constantly know the amount of tissue injected and the amount of tissue still available, particularly for reasons of overall symmetry and/or aesthetics, such as in the case of volume augmentation in two distinct areas (for example two breasts or buttocks).

A known technique of the prior art is to condition the adipose tissue intended for the injection in syringes having a determined volume.

Document US 2020/0054824 discloses a closed-loop system in which the fat is recovered to be prepared with a view to its reintroduction by being mixed with a washing liquid in a gravity filtration device marketed under the trade name Revolve®, and then to be reintroduced into the patient's body using a centrifugal pump.

However, none of the solutions of the prior art allows the practitioner to accurately assess and monitor the amount of fat to be removed during liposuction or to control easily and in real time the amount of injected and remaining purified fat.

### DISCLOSURE OF THE INVENTION

For this purpose, the present application proposes a fat transplantation method comprising:
the suction of an adipose tissue into a centrifugation chamber delimited by a filter, the filter having a pore size configured to allow a liquid medium to pass and retain an adipose tissue,
the filtration by centrifugation of a liquid medium present with the adipose tissue, the centrifugation being carried out continuously or sequentially when a predetermined mass of adipose tissue has been suctioned,
the calculation of an injectable amount of adipose tissue continuously or after each centrifugation sequence as a function of the mass of adipose tissue present in the centrifugation chamber,
the purification of the adipose tissue comprising the mixing of a washing liquid with the adipose tissue present in the centrifugation chamber and the filtration of a fraction of the polluting materials from the adipose tissue by centrifugation of the mixture,
the injection of the purified adipose tissue.

A method makes it possible to accurately determine by weighing and calculation of the amount of useful adipose tissue, i.e. the injectable amount of adipose tissue, and this from the raw adipose tissue removed during the suction. The practitioner can therefore monitor the evolution of the injectable amount of adipose tissue during suction and stop the suction at the right time, i.e. when he has reached the desired amount for the treatment. This ensures the removal of an optimal amount of fat.

According to one particular characteristic of a method disclosed herein, the injectable amount of adipose tissue (Q_{injectable}) is calculated with the following formula:

$${Q}_{injectable} = ( a \times {Q}_{total} - b )$$

where: a is a correction factor less than 1,
   Qₜₒₜₐₗ is the amount of adipose tissue measured after filtration,
   b is a loss factor.

Such a formula makes it possible to improve the accuracy of the calculation of the injectable amount because it takes into account in particular a loss factor related to the filtration device.

According to another particular characteristic of the method of the application, the latter further comprises the comparison of the calculated injectable amount of adipose tissue with a value of the target injectable amount of adipose tissue and a notification step when the calculated injectable amount of adipose tissue has reached the value of the target injectable amount of adipose tissue. This further facilitates the control of the amount of adipose tissue to be removed for the practitioner.

According to another particular characteristic of the method of the application, the latter further comprises the determination of the amount of purified adipose tissue available as a function of the measured mass of the purified adipose tissue and of a value of the lost mass of adipose tissue. This refines the assessment of the amount of tissue available before the injection.

According to another particular characteristic of the method of the application, it further comprises the real-time monitoring of the amount of purified adipose tissue available during the injection of the purified adipose tissue. The practitioner thus knows at all times the amount of adipose tissue available and can control the treatment very accurately.

The application also relates to a fat transplantation machine, the machine comprising at least:
a fat suction circuit,
a fat injection circuit,
a filtration device comprising a filter delimiting a centrifugation chamber for an adipose tissue, the filter having a pore size configured to allow a liquid medium to pass and retain an adipose tissue, the filtration device being connected to the fat suction circuit and to the fat injection circuit,
a rotational drive device comprising a rotary motor connected to the filtration device,
at least one washing liquid tank connected to the filtration device,
a mass measuring device configured to measure the mass of adipose tissue present in the filtration device,
a processing unit configured to control the rotational drive device so as to initiate at least one centrifugation filtration sequence as a function of a predetermined mass of suctioned adipose tissue, the processing unit being further configured to calculate an injectable amount of adipose tissue after each purification sequence.

The machine according to the application makes it possible to collect and filter the adipose tissue while allowing an accurate assessment of the amount of adipose tissue actually available for a subsequent injection. The fat suction can thus be stopped at the right time, i.e. when it has reached the desired amount for the treatment, which ensures a removal of an optimal amount of fat.

According to one particular characteristic of the machine according to the application, the processing unit calculates the injectable amount of adipose tissue from the following formula:

$${Q}_{injectable} = ( a \times {Q}_{total} - b )$$

where: a is a correction factor less than 1,
   Qₜₒₜₐₗ is the amount of adipose tissue measured after filtration,
   b is a loss factor.

Such a formula makes it possible to improve the accuracy of the calculation of the injectable amount because it takes into account in particular a loss factor related to the filtration device.

According to another particular characteristic of the machine according to the application, the processing unit is further configured to compare the calculated injectable amount of adipose tissue with a value of the target injectable amount of adipose tissue and to issue a notification when the calculated injectable amount of adipose tissue has reached the value of the target injectable amount of adipose tissue. This further facilitates the control of the amount of adipose tissue to be removed.

According to another particular characteristic of the machine according to the application, the processing unit is further configured to control the suction of the washing liquid into the filtration device and to control the rotational drive device so as to initiate at least one sequence of purification by filtration of a fraction of the polluting materials from the adipose tissue, the processing unit being further configured to determine the amount of purified adipose tissue available as a function of the measured mass of the purified adipose tissue and of a value of the lost mass of adipose tissue. The assessment of the amount of tissue available before the injection can thus be further refined.

According to another particular characteristic of the machine according to the application, the processing unit is configured to monitor in real time the amount of purified adipose tissue available during the injection of the purified adipose tissue.

The evolution of the amount of adipose tissue available can thus be controlled very accurately during the entire injection step.

### BRIEF DESCRIPTION OF THE DRAWINGS

FIG. 1 is a schematic perspective view of a fat transplantation machine;

FIG. 2 is an exploded schematic view of a filtration device of the machine of FIG. 1; and

FIG. 3 is a schematic cross-sectional view of a filtration device.

### DESCRIPTION OF THE EMBODIMENTS

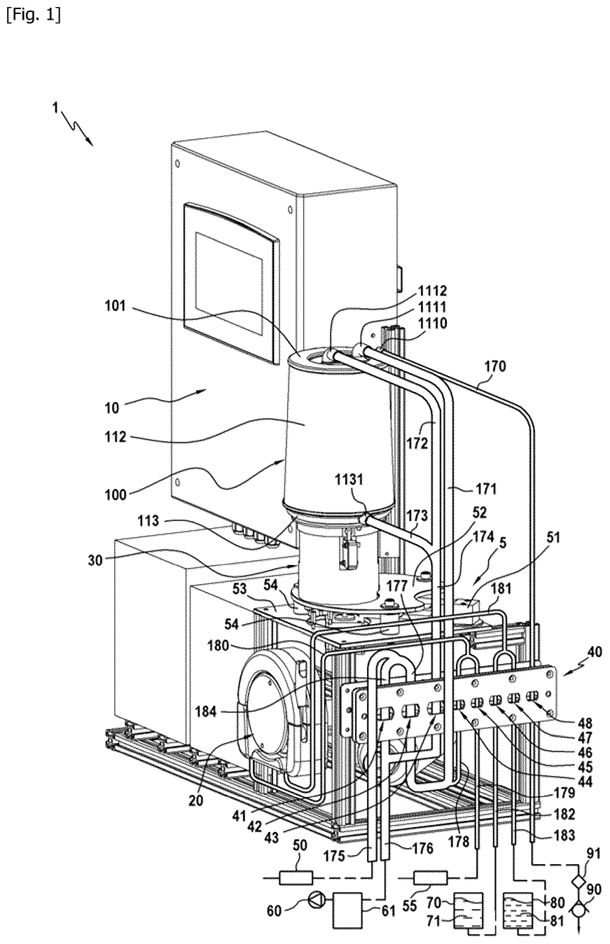

FIG. 1 illustrates one example of a fat transplantation machine 1. Generally and as will be detailed further below, the machine 1 comprises:
a filtration device 100,
a rotational drive device 30 connected to the filtration device,
a fat suction circuit comprising a suction cannula 50 and pipes 175, 177 and 171 connecting the suction cannula to the filtration device 100,
a fat injection circuit comprising an injection cannula 55 and pipes 179, 178 and 171 connecting the cannula to the filtration device 100,
a tank 70 containing a washing liquid 71 connected to the filtration device 100 by pipes 182, 181, 180, 178 and 171,
a tank 80 containing an infiltration liquid 81 connected to the injection cannula 55 by pipes 183, 181, 180 and 179,
a mass measuring device 5 connected here to the assembly comprising a part of the rotational drive device 30 and the filtration device 100,
a circulation pump 20,
a suction pump 60,
a pinch or throttle valve device 40,
a processing unit 10 allowing the implementation of the different fat transplantation steps, the processing unit 10 being connected to the rotational drive device 30, to the mass measuring device, to the circulation pump 20, to the suction pump 60 and to the pinch valve device 40.

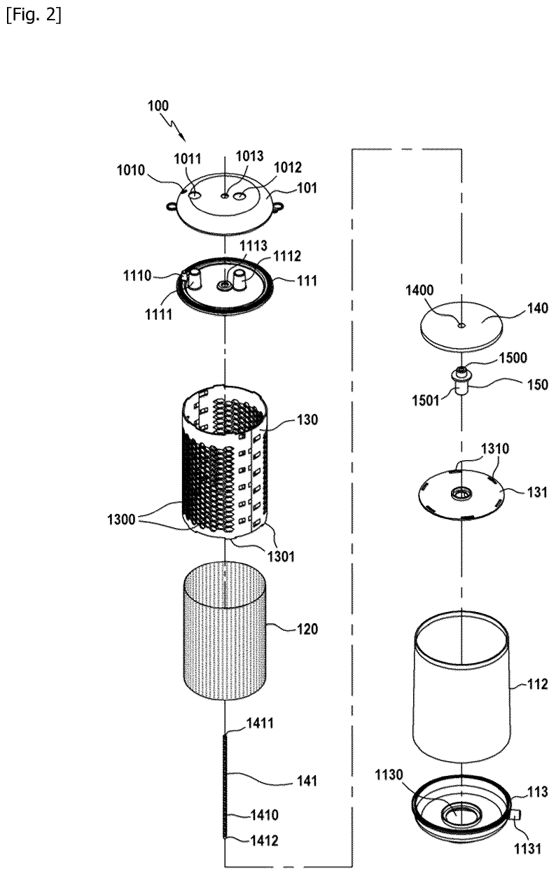

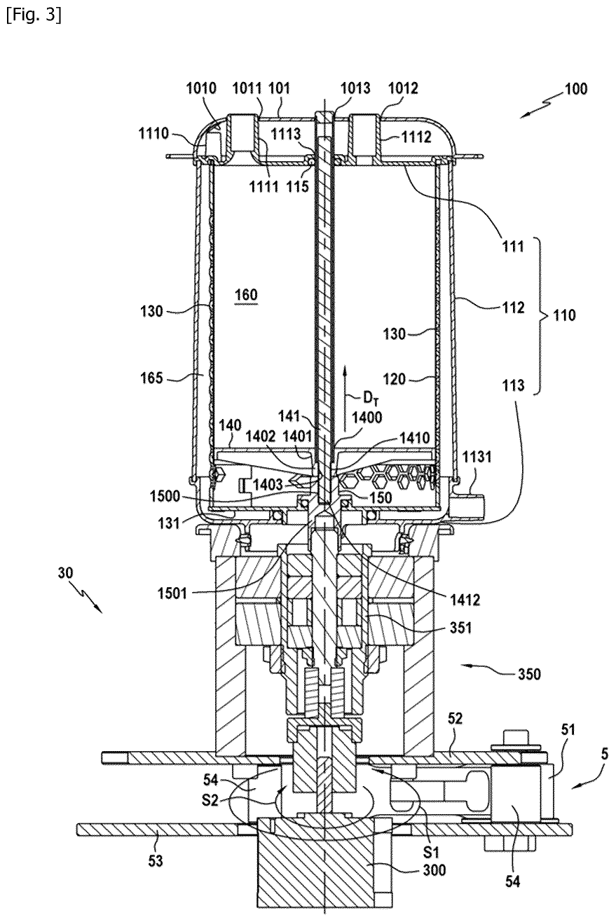

As illustrated in FIGS. 2 and 3, the filtration device 100 comprises a sealed enclosure 110 formed here by a cover 111, a cylindrical wall 112 and a bottom 113. These elements are fixed together in a sealed manner.

The purification device 100 also comprises a filter or sieve 120 present in the sealed enclosure 110. The filter 120 delimits a centrifugation chamber 160 (FIG. 3) whose operation is described later. In the example described here, the filter 120 has a cylindrical shape. The filter can have other shapes suitable for the centrifugation. The filter 120 has a pore size configured to allow a liquid medium to pass and retain an adipose tissue. The pore size is chosen particularly to allow the passage of liquids such as oil, blood, water or a physiological solution while retaining the adipose tissue. As non-limiting examples, the pore size of the filter can be comprised between 50 μm and 1,500 μm and more preferably between 200 μm and 500 μm.

In the example described here, a stiffening element 130 of cylindrical shape is present between the cylindrical wall 112 of the sealed enclosure 110 and the filter 120, the stiffening element 130 being for example made of metal or plastic material and having an openwork structure defining a plurality of openings 1300 in order to allow the discharge of the liquid medium drained by the filter 120.

The stiffening element 130 is associated with a rotary plate 131. More specifically, the stiffening element 130 includes, at its lower end, teeth 1301 which cooperate with grooves 1310 present in the vicinity of the outer periphery of the rotary plate 131. The rotary plate 131 is connected to the rotational drive device 30. The rotational drive device comprises a rotary electric motor 300. The electric motor 300 can be for example a stepper motor or a brushless DC motor. In the example described here, the rotary plate 131 is connected to the rotary electric motor 300 by a bidirectional clutch 350 configured to ensure, via a first shaft 351, the rotational drive of the rotary plate 131 along a first direction of rotation S₁ of the motor 30 (FIG. 3). The bidirectional clutch can be replaced by any device allowing a selective rotational drive along two opposite directions of rotation.

The centrifugation is performed by rotating the filter 120. More specifically, the electric motor 300 is controlled along a first direction of rotation S₁ to drive the rotary plate 131 and the stiffening element 130 engaged with the plate 131. Rotating the plate 131 and the stiffening element 130 causes the filter 120 to rotate. The filter is fixed to the stiffening element by any suitable means. The speed of the electric motor is controlled so as to apply a centrifugal acceleration to the adipose tissue present in the centrifugation chamber. Thus, an adipose tissue present in the centrifugation chamber 160 will be subjected to a centrifugal force against the inner wall of the filter 120, which makes it possible to effectively drain the liquid medium present in the adipose tissue without damaging it.

During the centrifugation, the liquid medium passing through the filter 120 and the stiffening element 130 is collected in a volume 165 delimited between the stiffening element 130 and the cylindrical wall 112 of the enclosure 110. The liquid is then discharged via a discharge port 1131 present on the bottom 113 of the enclosure 110 connected to a pipe 173.

In the example described here, the cover 111 comprises three ports 1110, 1111 and 1112 intended to be connected to a venting device 90 provided with a filter 91, to the fat injection circuit and to the tank 70 containing the washing liquid 71 via pipes 170, 171, 172.

The purification device comprises a cover 101 including openings 1010, 1011 and 1012 which cooperate with the ports 1110, 1111 and 1112 of the cover 111.

In the example described here, the purification device further comprises a collection plate 140 which is movable in translation in a direction D_{T} along the axis of the filter 120. More specifically, the purification device 100 comprises a threaded rod 141 which extends vertically inside the centrifugation chamber 160 along the axis of the filter 120. The lower end 1412 of the threaded rod is connected to the electric motor 300 via a guide 150 and the bidirectional clutch 350. The guide 150 comprises a housing 1500 in which the lower end 1412 is fixed. A lower portion 1501 of the guide 150 is connected to a part of the bidirectional clutch 350 which is engaged with the electric motor 300 only when the latter transmits a rotational movement along a second direction of rotation S₂ opposite to the first direction of rotation S₁ used for the centrifugation. When the electric motor 300 rotates along the first direction of rotation S₁, no rotational movement is transmitted by the bidirectional clutch 350 to the guide 150 to which the threaded rod 141 is connected. Similarly, when the motor 300 rotates along the second direction of rotation S₂, no rotational movement is transmitted by the bidirectional clutch 350 to the rotary plate 131 and, consequently, to the filter 120. The collection plate 140 includes on its upper face a central opening 1400 extended by a neck 1401 which extends from the lower face of the plate 140. The neck 1401 comprises a portion 1402 including a tapping 1403 which cooperates with a thread 1411 of the threaded rod 141. Thus, when the threaded rod is driven in rotation along the second direction of rotation S₂, the collection plate 140 rises in the centrifugation chamber 160 along the direction D_{T} along the direction D_{T} and acts as a piston.

In another exemplary embodiment, the filter is made of a self-supporting rigid material. The filter can be made in particular from a metal strip in which holes are made by laser, water jet or chemical cutting in order to form a sieve, a metal grid woven from wires or balls agglomerated by sintering of metal or ceramic powders. In this case, the stiffening element 130 is no longer necessary and it is the self-supporting filter which is directly engaged with the rotary plate 131.

In the example described here, the mass measuring device 5 corresponds to a strain gauge 51 interposed between a support plate 52 on which the assembly, comprising the bidirectional clutch 350 of the rotational drive device 30 and the filtration device 100 and a base plate 53 secured to the frame of the machine 1, rests. Spacers 54 are also interposed between the support plate 52 and the base plate 53, these spacers acting as safety stops in order to protect the strain gauge 51 from the untimely movements and/or overloads of the filtration device in a vertical and horizontal direction. Any other mass measuring device making it possible to measure the mass of the filtration device 100 or of the assembly formed by the rotational drive device 30 and the filtration device 100 could be used. The strain gauge 51 comprises a signal conditioner (not represented in FIGS. 1 and 2) which transforms an electric current representative of the mass measurement into a digital signal corresponding to the measured mass which is transmitted to the processing unit 10. The pinch valve device 40 comprises a plurality of pinch modules 41 to 48 also called “pinch valves” able to selectively pinch one or several pipes in order to prevent the circulation and release the pinch in order to authorize this circulation according to the commands received from the processing unit 10.

The processing unit 10 here has the hardware architecture of a computer. It includes in particular a processor, a random access memory, a read only memory and a non-volatile flash memory. The processing unit 10 also comprises means for communication with the rotational drive device 30, the mass measuring device 5, the circulation pump 20, the suction pump 60 and the pinch valve device 40. The communication means integrate for example digital data buses known per se, allowing various electronic entities to communicate with each other. The processing unit 10 further comprises a display screen 11.

The various steps of a fat transplantation method with the machine 1 will now be described. Before the implementation of each transplantation method, a preferably single-use filtration device 100 is connected to the rotational drive device 30 of the machine 1. A tare is then performed in order to deduce the mass of the part of the machine that rests on the weighing member, namely in the example described here the mass of the filtration device 100 and of the bidirectional clutch 350. This tare makes it possible to initialize the mass measurement to zero in order to subsequently measure only the mass of adipose tissue present in the filtration device. The tare can be performed automatically by programming the processing unit or by the practitioner, for example via a touch key on the display screen 11.

A first step that may be optional consists in injecting an infiltration liquid 81 containing physiological fluid with possibly an anesthetic into the area of the patient's body where adipose tissue is to be removed (infiltration). In this infiltration step, the processing unit 10 places the pinch modules 45 and 47 in the open position authorizing the circulation in the pipes 179 and 183 (the other pinch modules being placed in the closed position) and controls the actuation of the pump 20 so that the infiltration liquid 81 present in the tank 80 is suctioned by the pipe 183. The liquid is conveyed into the pump 20 by the pipe 181 and is then pushed back from the pump by the pipe 180 and the pipe 179 in order to reach the injection cannula 55 used to introduce the infiltration liquid into the patient's body.

The next step consists in suctioning an adipose tissue with a view to its subsequent reimplantation. In this step of removing the adipose tissue, the processing unit 10 places the pinch modules 42 and 43 in the open position authorizing the circulation in the pipes 177 and 174 (the other pinch modules being placed in the closed position). The actuation of the suction pump 60 by the processing unit 10 makes it possible to pull vacuum into the filtration device 100 via the pipes 176, 174, 173 and 172, which causes the suction of the adipose tissue by the suction cannula 50 and its conveying into the filtration device 100 via the pipes 175, 177 and 171.

The liquid medium present in the suctioned adipose tissue and mainly consisting of oil, blood and possibly infiltration liquid is filtered by centrifugation during the suction of the adipose tissue. The centrifugation can be carried out continuously or sequentially. In the latter case, centrifugation sequences are programmed in the processing unit 10 as a function of the mass of adipose tissue present in the centrifugation chamber 160 of the filtration device 100. More specifically, the processing unit 10 is programmed to initiate a centrifugation sequence as soon as a determined mass of adipose tissue has been suctioned into the filtration device, the processing unit receiving in real time a measurement of the mass of the adipose tissue present in the filtration device. As non-limiting examples, a centrifugation sequence can be initiated every 50, 100 or 150 grams of adipose tissue suctioned. Continuously or after each centrifugation sequence, the processing unit calculates the injectable amount of adipose tissue as a function of the mass of adipose tissue present in the centrifugation chamber.

The mass of injectable adipose tissue can be calculated with the following formula:

$${Q}_{injectable} = ( a \times {Q}_{total} - b )$$

where:
   a is a correction factor less than 1,
   Qₜₒₜₐₗ is the amount of adipose tissue measured after filtration,
   b is a loss factor.

The factor a can be determined by tests in which the amount of tissue weighed by the machine after filtration (Qₜₒₜₐₗ) is compared with the amount of tissue actually recovered after having completely emptied the centrifugation chamber.

The value of the amount Qₜₒₜₐₗ corresponds to the mass of adipose tissue measured by the mass measuring device 5 converted into volume, considering for example that a mass of 1 gram of adipose tissue corresponds to a volume of 1 milliliter of adipose tissue.

The factor b corresponds to a fraction of stagnant liquid in the filtration device (i.e. not discharged) and to a fraction of adipose tissue attached to the filter and which cannot be re-injected. The fraction of stagnant liquid is constant while the fraction of adipose tissue blocked in the filter depends on the centrifuged amount of adipose tissue. Values of the factor b can be determined by tests or by simulation. In the case of tests, a determined gross mass of adipose tissue is centrifuged. The amount of accessible adipose tissue is extracted from the filtration device. The mass of this extracted amount is measured and subtracted from the determined gross mass in order to obtain a value of lost mass. The conversion into volume of the lost mass then makes it possible to define the value of the loss factor b. It is thus possible to extrapolate a chart from several values of the loss factor obtained by tests. A non-limiting example of calculation is given below when a filtration is carried out by centrifugation after having suctioned 100 g of adipose tissue, 100 g corresponding here to the frequency of the centrifugation sequences:

The measured mass of the adipose tissue after centrifugation (elimination of the liquid phase present in the tissue) is of 85 g namely an amount Qₜₒₜₐₗ of 85 ml. The value of the correction factor a is set to 0.8 and the value of the loss factor b is set to 40 ml.

The injectable amount of tissue Q_{injectable} is therefore: (0.8×85)−40=28 ml.

The suction of the adipose tissue ends when the calculated injectable amount of tissue corresponds to the amount that the practitioner needs for the injection. The practitioner can himself monitor on the display screen 11 the evolution of the result of the calculation of the injectable amount of tissue and stop the suction when this amount suits him.

According to one particular aspect of the application, the processing unit 10 can be programmed to signal to the practitioner when the desired injectable amount of tissue is reached. To this end, the practitioner enters a value of the target injectable amount of adipose tissue, for example using touch keys on the display screen 11. During the suction of the adipose tissue, the processing unit 10 compares the calculated injectable amount of adipose tissue with the value of the target injectable amount of adipose tissue. Once the target amount has been reached, the processing unit notifies the practitioner, for example by an audible signal and/or information displayed on the display screen.

When the calculated injectable amount of tissue corresponds to the desired amount, the suction is stopped. The adipose tissue is then purified by washing and centrifugation. For this purpose, the processing unit 10 places the pinch modules 44 and 46 in the open position authorizing the circulation in the pipes 178 and 179 (the other pinch modules being placed in the closed position). Through the actuation of the pump 20 by the processing unit 10, a washing liquid 71 present in the tank 70 is suctioned by the pipe 182. The liquid is conveyed into the pump 20 by the pipe 181 and then pushed back from the pump by the pipe 180, the pipe 178 and the pipe 171 in order to reach the filtration device 100. During the introduction of the washing liquid into the filtration device 100, one or several centrifugation cycles are carried out as described above to filter the polluting materials. The discharge of the washing liquid containing the polluting materials (physiological fluid, blood, oil, etc.) from the adipose tissue is carried out through actuation of the suction pump 60 and placement of the pinch modules 41, 43 and 48 in the open position by the processing unit 10. In this configuration, the washing liquid containing the polluting materials is suctioned through the discharge port 1131 of the filtration device 100 and then circulates in the pipes 173, 174 and 176 in order to be conveyed to a bin 61 present upstream of the suction pump 60. The placement of the module 48 in the open position allows the venting of the filtration device and thus facilitates the discharge of the washing liquid containing the polluting materials. The purification of the adipose tissue preferably comprises two successive cycles, each comprising bringing the adipose tissue into contact with the washing liquid and centrifuging the mixture. Once the adipose tissue has been purified, the processing unit determines the amount of purified tissue available for the injection. This determination simply consists in measuring the mass of purified adipose tissue present in the filtration device 100, converting it into volume (amount) and subtracting from it the loss factor b defined for the considered amount of tissue. This gives an accurate value of the amount of adipose tissue available for the injection. This value is displayed on the screen 11.

The practitioner then injects the adipose tissue. The processing unit 10 places the pinch modules 44 and 45 in the open position authorizing the circulation of the adipose tissue in the pipes 171, 178 and 179 (the other pinch modules being in the closed position). The purified adipose tissue can thus be conveyed from the centrifugation chamber to the injection cannula 55.

The collection plate 140 can be used as a piston that pushes the purified adipose tissue toward the upper part of the centrifugation chamber as close as possible to the port 1111. A pump can also be interposed in the injection circuit to assist in conveying the adipose tissue to the injection cannula 55.

Throughout the injection, the mass of the adipose tissue present in the centrifugation chamber is transmitted to the processing unit 10. The processing unit calculates in real time the amount still available for the injection by comparison with the initial value of the available amount of adipose tissue. The amount of adipose tissue injected and/or the amount of adipose tissue still available is indicated on the display screen 11.

1. A fat process method comprising:
the filtration by centrifugation of a liquid medium present with an adipose tissue in a centrifugation chamber delimited by a filter, the filter having a pore size configured to allow a liquid medium to pass and retain an adipose tissue, the centrifugation being carried out when a predetermined mass of adipose tissue is present in the centrifugation chamber,
the calculation of an injectable amount of adipose tissue (Q_{injectable}) as a function of the mass of adipose tissue present in the centrifugation chamber, and
the purification of the adipose tissue comprising the mixing of a washing liquid with the adipose tissue present in the centrifugation chamber and the filtration of a fraction of the polluting materials from the adipose tissue by centrifugation of the mixture.

2. The method according to claim 1, wherein the injectable amount of adipose tissue (Q_{injectable}) is calculated with the following formula:
$${Q}_{injectable} = ( a \times {Q}_{total} - b )$$
where: a is a correction factor less than 1,
Qₜₒₜₐₗ is the amount of adipose tissue measured after filtration,
b is a loss factor.

3. The method according to claim 1, further comprising the comparison of the calculated injectable amount of adipose tissue (Q_{injectable}) with a value of the target injectable amount of adipose tissue and a notification step when the calculated injectable amount of adipose tissue has reached the value of the target injectable amount of adipose tissue.

4. The method according to claim 1, further comprising the determination of the amount of purified adipose tissue available as a function of the measured mass of the purified adipose tissue and of a value of the loss mass of adipose tissue.

5. The method according to claim 4, further comprising the real-time monitoring of the amount of purified adipose tissue available.

6. A fat transplantation machine comprising:
a fat suction circuit,
a fat injection circuit,
a filtration device comprising a filter delimiting a centrifugation chamber for an adipose tissue, the filter having a pore size configured to allow a liquid medium to pass and retain an adipose tissue, the filtration device being connected to the fat suction circuit and to the fat injection circuit, a rotational drive device comprising a rotary motor connected to the filtration device,
at least one washing liquid tank connected to the filtration device,
a mass measuring device configured to measure the mass of adipose tissue present in the filtration device, and
a processing unit configured to control the rotational drive device so as to initiate at least one centrifugation filtration sequence as a function of a predetermined mass of suctioned adipose tissue, the processing unit being further configured to calculate an injectable amount of adipose tissue (Q_{injectable}) after each purification sequence.

7. The machine according to claim 6, wherein the processing unit calculates the injectable amount of adipose tissue from the following formula:
$${Q}_{injectable} = ( a \times {Q}_{total} - b )$$
where: a is a correction factor less than 1,
Qₜₒₜₐₗ is the amount of adipose tissue measured after filtration,
b is a loss factor.

8. The machine according to claim 6, wherein the processing unit is further configured to compare the calculated injectable amount of adipose tissue with a value of the target injectable amount of adipose tissue and to issue a notification when the calculated injectable amount of adipose tissue has reached the value of the target injectable amount of adipose tissue.

9. The machine according to claim 6, wherein the processing unit is further configured to control the suction of a washing liquid into the filtration device and to control the rotational drive device so as to initiate at least one sequence of purification by filtration of a fraction of the polluting materials from the adipose tissue, the processing unit being further configured to determine the amount of purified adipose tissue available as a function of the measured mass of the purified adipose tissue and of a value of lost mass of adipose tissue.

10. The machine according to claim 9, wherein the processing unit is configured to monitor in real time the amount of purified adipose tissue available during the injection of the purified adipose tissue.

11. A processing unit that calculates an injectable amount of adipose tissue from the following formula:
$${Q}_{injectable} = ( a \times {Q}_{total} - b )$$
where: a is a correction factor less than 1,
Qₜₒₜₐₗ is the amount of adipose tissue measured after filtration,
b is a loss factor.
